(19) United States
(12) Patent Application Publication
(10) Pub. No.: US 2007/0254323 A1
(43) Pub. Date: Nov. 1, 2007

(54) Malachite green derivatives for immunoassay reagents to detect malachite green

(76) Inventors: Jun Wang, (US); Yunqing Han, (US); Bin Shen, Austin, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/788,396

(22) Filed: Apr. 19, 2007

Related U.S. Application Data
(60) Provisional application No. 60/745,358, filed on Apr. 21, 2006.

(51) Int. Cl. G01N 33/53; C07K 16/46

(52) U.S. Cl. 435/7.92; 530/409

(57) ABSTRACT

Compounds and procedures are provided for use in the preparation of reagents that can be used in immunoassays for Malachite Green, Leucomalachite Green and Carbinol. The compounds are Malachite Green Chromatic derivatives conjugated to several positions of the Malachite Green Chromatic structure, which in turn are conjugated to antigenic polypeptides for formation of antibodies for use in immunoassays. The antibodies produced, when employed in immunoassays with specified procedures, are found to be able to detect Malachite Green Chromatic as determined by positive results with fish tissue and water sample containing Malachite Green Chromatic that has been detected by HPLC or LC-MS.

### PRIORITY CLAIM

This application claims priority to U.S. Provisional Patent Application No. 60/745,358 entitled “MALACHITE GREEN DERIVATIVES FOR IMMUNOASSAY REAGENTS TO DETECT MALACHITE GREEN” filed on Apr. 21, 2006, which is incorporated by reference herein.

### BACKGROUND OF THE INVENTION

1. Field of the Invention

This present invention relates to antigen synthesis and corresponding antibody against Malachite Green used in novel immunoassays and extraction procedures to detect Malachite Green Chromatic, Leucomalachite Green and Carbinol. The antibodies produced, when employed in immunoassays, are found to be able to detect Malachite Green in fish tissue and water samples containing Malachite Green. The invention also relates to an immunoassay and kits for detecting and quantifying Malachite Green Chromatic, Leucomalachite Green and Carbinol in a sample.

2. Description of the Relevant Art

The triphenylmethane dye Malachite Green (Basic Green 4, CI 42000) has found extensive use all over the world in the fish farming industry as a fungicide, ectoparasiticide and disinfectant for treatment or prevention of external fungal and parasitic infections in fish and fish eggs (Alderman 1985; Manz et al. 1991; Sagar et al. 1994). Malachite Green is also effective as a systemic antiprotozoal agent in fish (Clifton-Hadley and Alderman 1987). However, the use of this compound in aquatic food animals is highly restricted or banned in many countries because of toxicological considerations. Malachite Green has been implicated as a teratogen (Werth 1958; Meyer and Jorgenson 1983) and tumor promoter in animals (Fernandes et al. 1991) and mutagenity (Culp and Beland 1996; Culp et al. 2002; Fessard et al. 1999). Malachite Green is also a respiratory enzyme poison (Werth and, Boiteux 1968a). Malachite Green is typically administered as a bath treatment, alone or in combination with formalin. In aqueous solution, the chromatic form of Malachite Green (MG-C) combines slowly with available hydroxyl ions to produce the non-ionized and colorless carbinol form (Alderman 1985) (FIG. 2). The carbinol form is more lipid soluble than MG-C, which may favor its passage across cellular membranes. Malachite Green is readily absorbed by fish and fish eggs during waterborne exposure (Nakagawa et al. 1984; Bauer et al. 1988; Kietzmann et al. 1990; Kasuga et al. 1992; Alderman and Clifton-Hadley 1993). Residues have also been found in eggs collected from adult fish treated with Malachite Green (Allen and Hunn 1986; Allen 1990). The reduced derivative Leucomalachite Green (LMG) is generally recognized as a major metabolite of Malachite Green in animal tissue (FIG. 2) (Werth and Boiteux 1968b; Poe and Wilson 1983; Bauer et al. 1988; Allen et al. 1994; Plakas et al. 1995; Roybal et al. 1995).

The presence of Malachite Green residues in animal tissue is a concern for food safety, especially when the compound has been used illegally or in a manner proscribed by regulatory officials (off-label use). In an effort to combat the illicit use of Malachite Green, regulatory organizations worldwide test animal tissue or excreta for the presence of Malachite Green. An increasing number of methods have been developed in recent years for their detection. Several papers described the application of new analytical procedures for the detection and determination of Malachite Green and Leucomalachite Green form in various matrices, such as water, urine, fish tissue, animal feed, etc. (Rushing and Bowman 1980; Rushing and Hansen 1997; Sagar et al. 1994; Rushing et al. 1995). In most cases, liquid chromatography procedures were used for the analysis and the dyes usually had to be preconcentrated from the analyzed samples using various extraction procedures. Although the liquid chromatography (LC) methods such as HPLC or LC-MS are accurate, they are extremely expensive and time-consuming for sample preparations.

To prevent Malachite Green residues from entering the food chain, both producers and government surveillance organizations need not only confirmatory assays such as HPLC and LC-MS, but also rapid screening methods that provide rapid, accurate results that are also reliable at the required sensitivity. Enzyme Immunoassays (ELISA) are convenient screening tools used to detect the presence of an analyte in various matrices, and have wide application for determination of the presence of environmental toxins (Sanborn et al. 1998), herbicides (Clegg et al. 1999), insecticides/pesticides (Wang et al. 1999; Abad et al. 1999), and pharmaceuticals (Brandon et al. 1998). A successful screening assay should be quick, reliable, and relatively inexpensive. Positive samples from screening assays may then be assayed by more costly and complex instrumental methods such as HPLC or LC-MS that unequivocally identify the analyte in the sample (eliminating false-positives). However, for screening, immunoassays provide the advantages of high throughput, portability, and sensitivity (detection limits in the ppb range). High sensitivities of immunoassays are particularly desirable for off-label drug monitoring because it may be desirable to detect the analyte even after extended withdrawal periods.

Development of immunoassays for Malachite Green Chromatic, Leucomalachite Green and Carbinol will enable food manufacturers and quality assurance organizations to rapidly detect these substances in fish and shrimp matrices and to satisfy customer concerns for food safety. Due to the strong need for such a test, we developed immunoassay and antibodies for detecting Malachite Green Chromatic, Leucomalachite Green and Carbinol and extraction methods for these compounds for testing purposes.

In producing antibodies, one normally conjugates a molecule that resembles the compound of interest to a large molecule that is antigenic. The resulting conjugate is then injected into an animal, normally a domestic animal, to elicit an immunological response with the production of antibodies that recognize the compound of interest.

In producing antibodies, there are a number of considerations as to the usefulness of the antibody composition in an immunoassay. The concentration of useful antibodies must be sufficiently high, so that upon dilution in the assay, there is sufficient antibody to provide for the necessary sensitivity. The binding constant of the antibody should also be high, so that large concentrations of antibodies are not required to ensure a reasonable amount of binding of any of the compound present to the antibody. It should be recognized that normally the concentrations of interest are only a few nanograms per milliliter or less so that one is dealing with extremely small amounts of the compound being assayed.

A patent application (US 2007/0072242) entitled Immunoassay Methods and kit to Leucomalachite Green and Malachite Green Chromatic from Randox Laboratories, Limited was published Mar. 29, 2007. In this application, antibodies and assays to detect Malachite Green Chromatic and Leucomalachite Green have been demonstrated. The significant difference with the assay developed by Randox and the one described herein are several fold. First, Randox uses Leucomalichite Green as the immunogen. Second, the antibody developed from this immunogen cross-reacts with Malachite Green Chromatic. This antibody cross reactivity requires that standard curves for both Malachite Green Chromatic and Leucomalachite Green are included in the assay providing less room for sample analysis per microtiter plate as compared to the assay developed by BIOO Scientific. Second the cross reactivity between the Leukomalachite Green antibody with Malachite Green Chromatic is only 26.8% percent. This differential antibody reactivity between Malachite Green Chromatic and Leukomalachite Green complicates the analysis and quantification of total amounts of substance in the sample. Contrary, BIOO Scientific developed antibody using Malachite Green as the immunogen and the antibody generated is specific for Malachite Green Chromatic with minimal cross or no reactivity to Leukomalachite Green and Carbinol. BIOO Scientific then developed novel assay and sample extraction methods and procedures so that 100% of the Leucomalichite Green and Carbinol are converted to Malachite Green in 5 minutes. Due to the efficiency of the conversion of Leucomalachite Green and Carbinol to Malachite Green Chromatic and the specificity of our antibody for Malachite Green Chromatic the BIOO Scientific assay and kit have greater utility than the Randox assay. BIOO Scientific kits only require a standard curve for Malachite Green Chromatic and due to the efficient conversion of Leukomalachite Green and Carbinol to Malachite Green Chromatic it will prove to be more accurate and sensitive of an assay than the Randox assay. In addition, the BIOO Scientific Malachite Green Chromatic assay kit is already being used in over 20 countries worldwide.

In the present invention, we report the method to synthesize the antigen and produce antibodies against Malachite Green. The antibodies produced, when employed in immunoassays, were found to be able to detect Malachite Green, Leucomalachite Green and Carbinol as determined by positive results with fish tissue and water sample containing Malachite Green, Leucomalachite Green and Carbinol that has been detected by HPLC or LC-MS.

### SUMMARY OF THE INVENTION

Compounds are provided for use in the preparation of reagents that can be used in immunoassays for detecting and quantifying Malachite Green. The compounds are Malachite Green derivatives conjugated to several positions of the Malachite Green structure, which in turn are conjugated to antigenic polypeptides for formation of antibodies for use in immunoassays. Conjugating antigenic polypeptides to Leucomalachite Green and Carbinol are also contemplated. The antibodies produced, when employed in immunoassays, are found to be able to detect Malachite Green as determined by positive results with the fish tissue and water sample containing Malachite Green that has been detected by HPLC or LC-MS.

The antibody of this invention may be incorporated into kits for the detection and quantitative determination of low levels of Malachite Green in samples, especially in food, feed, animals, animal excreta, human tissue, human excreta, plants, soil, air and water. Detection of Malachite Green in sample materials is accomplished using immunosorbent assay procedures conventional in the art with special considerations taken to convert Leucomalachite Green and Carbinol to Malachite Green Chromatic.

Another object of this invention is to provide immunoassay methods for determining off-label and illegal use of Malachite Green and for detecting levels of Malachite Green Chromatic, Leucomalachite Green and Carbinol exceeding tolerance limits in samples.

A further object is to provide kits useful for the assay of Malachite Green Leucomalachite Green and Carbinol based on the antibody and methods described herein. Yet another object is to provide a method for recovering or removing Malachite Green Chromatic, Leucomalachite Green and Carbinol from any material such as by affinity purification. Other objects and advantages of this invention will become readily apparent from the ensuing description.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a competitive ELISA standard curve for relative absorbance (%) against different concentrations of Malachite Green standards.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

In accordance with this invention we have created the antibodies that are prepared produced in response to the conjugated antigens of this invention; have strong specific binding to Malachite Green, the conjugated antigen and the compound or derivative thereof used to conjugate to the antigen. The antibodies are effective for detecting and quantifying very low levels of Malachite Green Chromatic, Leucomalachite Green and Carbinol. The antibodies of the invention are capable of detecting Malachite Green Chromatic, Leucomalachite Green and Carbinol at the level of 0.5 ng/mL in competitive inhibition ELISA. Due to the similarity of Malachite Green with Leucomalachite Green and Carbinol it is contemplated that antibody specific for these compounds can be generated using a similar approach as described for Malachite Green. 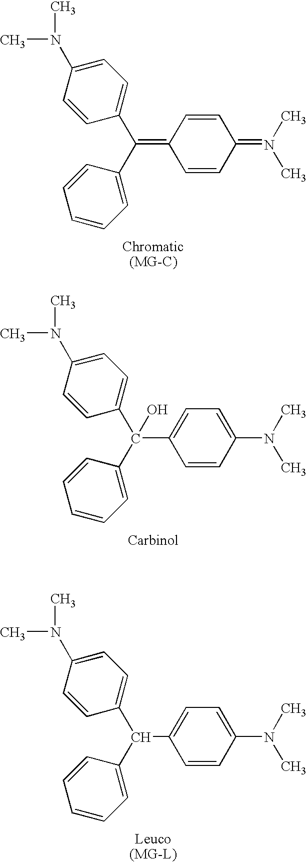
The chemical structures of Malachite Green (Chromatic form, MG-C) and its derivatives Carbinol and Leucomalachite Green (Leuco form, MG-L).

Generally, the process of preparation of monoclonal antibodies comprises the steps of immunizing an animal with the antigen of interest, recovering splenocytes or lymphocytes from the animal, fusing the splenocytes or lymphocytes with continuously replicating myeloma cells to produce hybrid cells, and screening the resultant hybrid cells for the production of antibodies to the antigen. For preparation of polyclonal antibodies, the process involves immunizing an animal such as a rabbit with the antigen of interest for a period of time (6-8 weeks), and testing the antiserum's titer by competitive inhibition ELISA. Because Malachite Green is a relatively small molecule, it is itself incapable of stimulating the immune system to produce antibodies. To render the compound immunogenic, it must first be conjugated to an immunogenic carrier in such a manner that the resultant immunogen is capable of stimulating the immune system of an animal to produce specific antibodies that are capable of binding the unconjugated Malachite Green Chromatic.

The structure of Malachite Green Chromatic is shown below. This compound was rendered immunogenic by coupling it to an immunogenic carrier by the following procedure.

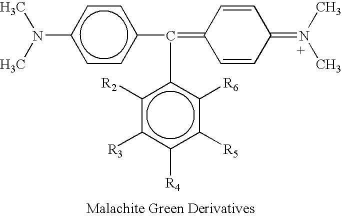

The immunizing agent was constructed by covalently conjugating Malachite Green Chromatic to an immunogenic carrier protein, preferably by means of a crosslinker, such as —N═C═S. Immunogenic carriers are defined herein as any compound to which Malachite Green Chromatic may be attached to render it immunogenic. Suitable carriers are well known and may be readily determined by the skilled practitioner in the art. Without being limited thereto, preferred carriers include proteins such as keyhole limpet hemocyanin (KLH), bovine serum albumin (BSA), ovalbumin (OVA) and human thyroglobulin. The function of the crosslinker is to introduce into the molecule a spacer of sufficient size to prevent the carrier protein from masking the Malachite Green Chromatic molecule.

Antibodies were prepared in response to an antigen of Malachite Green Chromatic conjugating with antigenic poly(amino acid) via a linker R4 or any of R2, R3, R5, and R6. Wherein R (present in any of R2, R3, R4, R5, R6) is a linking group of 0 to 8 carbon atoms, 0-16 hydrogen atoms, 0 to 3 nitrogen atom bonded to carbon, 0-4 oxygen atom bonded to carbon and 0-1 sulfur bonded to carbon. One example of the crosslinker is —N═C═S. at R4 position. The conjugating number of Malachite Green Chromatic derivatives per poly (amino acid) is at least 1 but no greater than the molecular weight of the poly (amino acid) divided by 500.

To produce antibodies, an immunizing preparation comprising the antigen (Malachite Green Chromatic derivative-KLH conjugate) was injected into an immunologically competent animal. The preparation may also contain other proteins or Malachite Green Chromatic derivative carrier conjugate, although pure or substantially pure compositions of the conjugate in a pharmaceutically acceptable carrier are preferred. Without being limited thereto, mice, rats and particularly rabbits are preferred hosts for raising antibodies because of ease of handling. The injection procedures are well known in the literature.

Certain aspects of the immunization protocol are used to produce high affinity antibodies. It is advantageous that the injection schedule is sufficient to allow for maturation of the immune response in the animal. Preferably, a series comprising an initial injection followed by 4-6 boosters at approximately 60-day intervals should be given to achieve a high titer. The dose of antigen injected should, of course, be sufficient to stimulate the immune system. Extrapolating from the success of employing 400 μg conjugate per injection in the Examples below, it is anticipated that any reasonable amount in excess of about 50 μg per injection would also be effective. Typically, the antigen is suspended in a physiological vehicle, such as isotonic saline or PBS buffer. In accordance with a preferred embodiment, an adjuvant may be incorporated into the immunizing preparation for enhanced stimulation of the immune system, particularly for the first 1-2 injections. A variety of adjuvants, which are conventional in the art, may be used herein, although Freund's or RIBI adjuvants are preferred. The route of injection is typically intraperitoneally, though other routes would also likely be effective in inducing an immune response.

To test the antiserum titer, in accordance with the preferred embodiment of the invention, a competitive indirect ELISA (CI-ELISA) is preferred, and is conducted at a final antibody concentration (dilution from antiserum) to give 50% of maximal binding to a Malachite Green Chromatic coated substrate or assay well. In accordance with this embodiment, the diluted antiserum is added to a Malachite Green Chromatic conjugate-coated solid substrate such as the wells of an assay plate, together with a range of concentrations of free Malachite Green Chromatic as a competitor. Following incubation and washing, bound antibody in the wells is determined. Percent inhibition may be calculated as (1−B/B₀)×100 where B is the optical density (OD) of a well with a competitor and B₀ is the mean OD of the wells without competitor (control). The relative affinity of the antibodies may be accurately measured as the concentration of free Malachite Green Chromatic added to the wells that resulted in at least 20% inhibition (IC₂₀) of the control activity. However, for even greater accuracy, the affinity may be alternatively measured at 50% inhibition (IC₅₀). A preferred coating is Malachite Green Chromatic derivative-OVA. Detection of bound antibody may be accomplished by addition of enzyme-labeled anti-immunoglobulin antibodies followed by enzyme substrate. Horseradish peroxidase and its substrate TMB are preferred enzyme/substrate labels. However, it is understood that other enzyme/substrate labels or non-enzyme labels such as radiolabels or chromophores may also be used.

To produce a large amount of antiserum, rabbits or sheep can be used. The antiserum can be directly used in ELISA. It may also be purified, for example, by affinity chromatography on a protein A or G resin, or using Malachite Green Chromatic bound to a resin.

The antibodies produced in accordance with this invention possess very high affinity for malachite Green Chromatic, allowing the determination of Malachite Green Chromatic at very low levels of less than 1 ng/mL, and even at levels of about 0.5 ng/mL or lower.

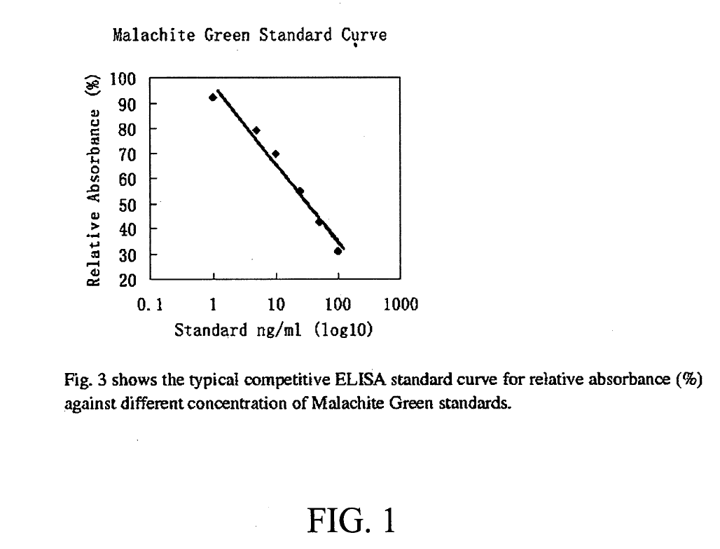

These antibodies may be used to detect and quantify Malachite Green Chromatic in unknown samples using the immunosorbent assay procedures including but not limited to RIA or ELISA. A competitive inhibition ELISA is preferred. In this assay, a sample to be analyzed is incubated with the antibodies for Malachite Green Chromatic and a solid substrate coated with Malachite Green Chromatic conjugate. It is preferred to use a conjugate having a protein different from that used in the immunization protocol in order to avoid detection of antibodies to the protein carrier. After incubation, the solid phase is drained and washed, bound antibody on the coating antigen is detected, and percent inhibition is calculated as described earlier. The concentration of Malachite Green Chromatic in the sample may then be determined by reference to a standard curve constructed from assays using known levels of Malachite Green Chromatic, as depicted in FIG. 1.

In one alternative embodiment, Malachite Green Chromatic may be determined by a competition ELISA using the antibodies of the invention attached to a solid support. For example, the anti-Malachite Green Chromatic antibody may be immobilized on a bead or in a microtiter well. The unknown sample to be analyzed, (or analytical standards of Malachite Green Chromatic) are then added together with enzyme-labeled, or radioactive labeled, Malachite Green Chromatic. The amount of labeled Malachite Green Chromatic bound to the antibody is then measured, using a substrate when the label is an enzyme. The amount of Malachite Green Chromatic in the sample is inversely proportional to the amount of bound labeled Malachite Green Chromatic. In another embodiment, the antibodies may be attached to a solid support for use in conventional double-antibody sandwich ELISA procedures.

An assay using the antibodies of the invention would have the advantages of being more specific than a direct spectrophotometer assay, and more rapid and less expensive than HPLC or LC-MS. It can be incorporated into a residue-monitoring program as a rapid initial screen to eliminate samples that do not contain levels of Malachite Green Chromatic that violate regulations. Therefore, use of this method to detect Malachite Green Chromatic in edible tissue or excretions has the potential to increase sample throughput and to decrease cost associated with sample analyses.

With any of the above-described assay formats, the antigens and antibodies of the invention may be incorporated into kits alone, or preferably together with any other necessary reagents. Such a kit for use herein comprises a first container comprising the antibodies, a second container comprising a detection reagent effective for detecting bound antibody, and Malachite Green Chromatic conjugate bound to a solid support.

Determination of Malachite Green Chromatic, Lecomalachite Green and Carbinol in animal tissue samples, body fluids, excretions or feeds may be conducted using the above-described assay with minimal sample preparation. Samples need to be homogenized and extracted with acetonitrile followed by a liquid-liquid extraction to partition the residue into dichloromethane. The dichloromethane is removed by roto-evaporation or hot nitrogen gas. Dissolve the residue of acetonitrile and dilute the sample with 1×PBS. Chromatic Malachite Green can be rapidly converted into colorless Leucomalachite Green (LMG) in fish (˜80%). To detect LMG by ELISA, LMG can be conversed to Malachite Green Chromatic before detection by adding oxidant agent such as lead oxide, 2,3-dichloro-5,6-dicyano-1,4-benzoquinone (DDQ). Chromatic Malachite Green is gradually degraded to colorless Carbinol in aqueous solution at pH 7. However, when adjust pH to 4.5 the Carbinol will be converted back to Chromatic Malachite Green. In the sample extraction procedure, pH4.5 acetate buffer is used to convert the Carbinol to Malachite Green. Therefore, the ELISA assay developed by this method can detect all three of MG, LMG and Carbinol.

Once Carbinol and Leucomalachite Green are converted to Malachite Green Chromatic the sample is ready for ELISA test. Although any animal tissue may be analyzed, the assay is particularly valuable for the determination of Malachite Green Chromatic in edible tissue. Tissue for analysis in accordance with the invention may originate from virtually any animal or environmental sample. Without being limited thereto, the analysis of tissue samples from fish, shrimp, water sample of fishbowls or fishpond, feed and other animal tissue are encompassed by this invention.

Another application of the antibodies is affinity purification of Malachite Green Chromatic. The antibodies may be bound to a matrix, column, or other support using well-known techniques and used to recover or remove Malachite Green Chromatic from any desired material. Alternatively, the antibodies may be incorporated into sensors such as solid phase electronic devices for detection of Malachite Green Chromatic in sample materials.

The following examples are intended only to further illustrate the invention and are not intended to limit the scope of the invention that is defined by the claims.

### EXAMPLE 1

### Malachite Green Chromatic KLH Conjugate

5 mg Malachite Green isothiocyanate in dimethyl sulfoxide were added to 10 mg of KLH in 2 ml 200 mM NaHCO₃ (pH 9.0). After 2 hr of incubation at room temperature with agitation, free Malachite Green isothiocyanate was separated from conjugated KLH by gel filtration. The ratio of Malachite Green isothiocyanate conjugating with KLH was determined by measuring the optical density at 620 nm of a solution of known KLH concentration and calculating the dye concentration by using a molar absorptivity ε=150,000 M⁻¹ .cm⁻¹.

### EXAMPLE 2

### Polyclonal Antibody Production

Two female New Zealand White Rabbits, 8 weeks old, were initially immunized by intraperitoneal injection with 0.2 mg of Malachite Green-KLH conjugate as prepared in EXAMPLE 1 mixed with complete Freund's adjuvant. Five booster immunizations were administered at 15-day intervals using the antigen. Blood was collected 7 to 10 days after the last booster immunization in order to check for antibody titers. Titers were checked using both indirect ELISA and competitive ELISA that employed Malachite Green Chromatic-BSA conjugate as the coating antigen and free Malachite Green Chromatic as competitor. One rabbit that produced high titers (1:40,000) after the 5^{th} boost, and showed competition toward Malachite Green Chromatic was used for further study. A typical competitive ELISA procedure is described below:
(1). In the Malachite Green Chromatic-BSA coated 96-well plate, add 50 μL of negative control and each Malachite Green Chromatic standard (1, 5, 10, 25, 50, 100 ng/mL) in duplicate into different wells; add 50 μL of each sample in duplicate into different sample wells.
(2). Add 100 μL of diluted Malachite Green Antibody #1 and mix well by gently tapping the edge of the plate for 10 seconds.
(3). Incubate the plate for 30 min at 37° C. or 1 hr at room temperature (15-25° C.).
(4). Wash the plate 3 times with 250 μL 1×PBS. After the last wash, gently slap the plate dry on paper towels.
(5). Add 150 μL of HRP Goat-against Rabbit antibody conjugate solution. Incubate the plate for 30 min at 37° C. or 1 hr at room temperature (15-25° C.).
(6). Wash the plate 3 times with 250 μL 1×PBS. After the last wash, gently slap the plate dry on paper towels.
(7). Add 100 μL of TMB substrate.
(8). Time the reaction immediately after adding the substrate; mix the solution by gently tapping the edge of the plate.
(9). After incubating for 15 min at room temperature, add 100 μL of 1 M H₂SO₄ to stop the enzyme reaction before reading the plate at 450 nm.

### EXAMPLE 3

### The Sensitivity and Specificity of the Produced Anti-Malachite Green Chromatic Antibodies

The sensitivity and specificity of the produced antibodies against Malachite Green Chromatic and its analogs were compared as in Table 1. The data suggest that the produced antibodies have as high as 1 ppb test sensitivity and specifically bind to Malachite Green Chromatic.

TABLE 1
The sensitivity and cross-reactivities of the
produced anti-Malachite Green antibodies.

| Analyte | Sensitivity (ng/ml) | Cross-reactivities (%) |
|---|---|---|
| Malachite Green | 1 | 100 |
| Crystal Violet | 10 | 42 |
| Leucomalachite Green | 500 | 1 |
| Leucocrystal Violet | 5000 | <0.01 |
| Clenbuterol | >10,000 | <0.01 |
| Sulfumethazine | >10,000 | <0.01 |
| Chloramphenicol | >10,000 | <0.01 |
| Furazolidone | >10,000 | <0.01 |

### EXAMPLE 4

### The Reproducibility of the ELISA Test Using the Produced Anti-Malachite Green Chromatic Antibodies

Table 2 shows that the different concentrations of Malachite Green Chromatic gave a similar low CV value (<5%) for ELISA test. The data suggest that it is practical to develop a Malachite Green Chromatic ELISA test kit.

TABLE 2
The precision of ELISA test for Malachite Green standard.

| Standard (ng/ml) | Replicates | *CV(%) |
|---|---|---|
| 0 | 8 | 2.3 |
| 1 | 8 | 2.3 |
| 5 | 8 | 2.3 |
| 25 | 8 | 2.5 |
| 50 | 8 | 2.9 |
| 100 | 8 | 3.6 |

### EXAMPLE 5

### Validation of Malachite Green Chromatic ELISA Results by LC-MS Method

Different amounts of Malachite Green Chromatic (MG-C) and Leucomalachite Green (LMG) were spiked in fish tissue as shown in Table 3. The fish tissue was extracted with following procedures.
(1) Blend boneless fish tissue in blender/homogenizer (e.g. Omni TissueMaster Homogenizer) until the material had consistency of fine powder. Store the sample in freezer (−20° C.) until analysis.
(2) Take 5 g homogenized fish/shrimp sample, add 1.5 mL of acetate buffer (pH 4.5) and 10 mL of acetonitrile. Vortex the sample for 3 min at maximum speed (e.g.14216-182 Vortex-Genie mixer, VWR).
(3) Centrifuge for 10 min at 4000 g at room temperature. Transfer the supernatant to a new tube containing 10 mL of water and 2 mL of diethylene glycol. Perform a second extraction: add another 5 ml of acetonitrile to the pellet, vortex the sample for 3 min at maximum speed, centrifuge for 10 min at 4000 g, and add the resulting supernatant to the first.
(4) Add 10 mL dichloromethane to the supernatant. A liquid-liquid extraction to partition the residue into dichloromethane is done by vigorously shaking the tube.
(5) Centrifuge the sample for 10 min at 4000×g and take out the lower dichloromethane layer. Perform a second extraction with another 5 mL of dichloromethane, and add the resulting dichloromethane layer to the first.
(6) Remove the solvent by roto-evaporation at reduced pressure or hot nitrogen gas at 70±5° C. Dissolve the residue in 0.2 mL of acetonitrile, vortex for 1 min at maximum speed. To convert Leucomalachite Green to Malachite Green Chromatic either pass the dissolved residue through a PbO₂ column or add 0. ml of 1 μM DDQ to the solution and react for 5 minutes at room temperature.
(7) Take 0.1 mL eluate, add 0.9 ml 1×PBS. The sample is ready for ELISA test.
(8) The un-diluted sample can be used for LC-MS validation

The ELISA method is described above. The LC-MS analysis was performed using a US FDA reported method (Tumipseed SB et al. Laboratory Information Bulletin, 20(11), 2004). The data suggest that the Malachite Green ELISA result has high consistency with the LC-MS results.

TABLE 3
Validation of Malachite Green (MG) ELISA results
by LC-MS method for fish tissue sample

| Spiked | | | |
|---|---|---|---|
| MG/LMG | Repli- | Test results from MG | Test results from |
| (1:1, ppb) | cates | ELISA kit (ppb) | LC-MS (ppb) |
| 0 | 10 | 0.02/0.11/0.08/0.23/0.12 | 0.04/0.18/0.08//0.11/0.45 |
| | | 0.25/0.41/0.23/0.15/0.52 | 0.33/0.21/0.39/0.65/0.17 |
| | | Ave. 0.21 ± 0.15 | Ave. 0.26 ± 0.19 |
| | | False Positive No: 0 | |
| | | False Negative No. 0 | |
| 1 | 10 | 0.91/0.88/0.95/0.90/0.89 | 0.96/0.84/0.91/0.87/0.84 |
| | | 0.88/0.86/0.97/0.98/1.12 | 0.86/0.84/0.95/0.88/0.87 |
| | | Ave. 0.93 ± 0.08 | Ave. 0.88 ± 0.04 |
| | | False Positive No. 1 | |
| | | False Negative No. 0 | |
| 2 | 10 | 1.80/1.84/1.72/1.89/1.75 | 1.78/1.80/1.70/1.82/1.72 |
| | | 1.99/1.82/1.81/1.81/1.71 | 1.90/1.72/1.80/1.84/1.73 |
| | | Ave. 1.81 ± 0.08 | Ave. 1.78 ± 0.06 |
| | | False Positive No. 0 | |
| | | False Negative No. 0 | |
| 3 | 10 | 2.80/2.81/2.72/2.54/2.62 | 2.70/2.61/2.62/2.62/2.41 |
| | | 2.58/2.82/2.85/2.87/2.71 | 2.67/2.74/2.61/2.77/2.72 |
| | | Ave. 2.73 ± 0.11 | Ave. 2.65 ± 0.10 |
| | | False Positive No. 0 | |
| | | False Negative No. 0 | |
| 4 | 10 | 3.60/3.71/3.42/3.54/3.62 | 3.50/3.62/3.38/3.45/3.49 |
| | | 3.48/3.82/3.75/3.57/3.61 | 3.44/3.62/3.55/3.67/3.51 |
| | | Ave. 3.61 ± 0.12 | Ave. 3.52 ± 0.09 |
| | | False Positive No. 0 | |
| | | False Negative No. 0 | |
| 5 | 10 | 4.60/4.41/4.22/4.54/4.42 | 4.51/4.22/4.12/4.44/4.38 |
| | | 4.48/4.82/4.15/4.47/4.41 | 4.39/4.71/4.25/4.23/4.46 |
| | | Ave. 4.45 ± 0.19 | Ave. 4.37 ± 0.17 |
| | | False Positive No. 0 | |
| | | False Negative No. 0 | |

False Positive Rate: 2% (50 samples)
False Negative Rate: 0% (50 samples)
Correlations between ELISA and LC-MS: y = 0.97x + 0.015; r² = 0.99 (y: LC-MS; x: ELISA)
Notes:
a) False Negative Result: if the LC-MC detection result is more than 1 ppb and the ELISA detection result is less than 1 ppb. this result is defined as False Negative Result
b) False Positive Result: if the LC-MC detection result is less than 1 ppb and the ELISA detection result is more than 1 ppb. this result is defined as False Positive Result

### EXAMPLE 6

### Method of Extracting Malachite Green Chromatic, Leucomalachite Green and Carbinol from Food Samples in Which a Differential Freezing is Obtained Between the Organic and Aqueous Phase of the Solution.

1. Blend boneless fish/shrimp tissue in a blender/homogenizer until the sample has consistency of fine paste. Store the sample at −20° C. or in a freezer till analysis.
2. Take 2.0 g of homogenized fish/shrimp sample, add 1 mL of 0.05 M Acetate Buffer (pH 4.5) and 6 mL of acetonitrile. Vortex for 3 minutes at maximum speed or rotorack for 20 minutes (e.g. VWR Tube Rotator).
3. Centrifuge the sample for 5 minutes at 4,000×g at room temperature (20-25° C./68-77° F.). Transfer 6 mL of the supernatant to a 15-mL tube containing 3.5 mL of water and 1.5 mL of dichloromethane, vigorously vortex the tube for 1 minute.
4. Centrifuge the sample for 5 minutes at 4,000×g, take out 4 mL of the upper organic solvent layer (The total upper layer is ˜5 mL, avoid the lower aqueous layer) To a new tube containing 0.1 mL of 1× Oxidant Solution, Keep the sample at room temperature (20-25° C./68-77° F.) for 5 minutes and then store the sample at -20° C. or in a freezer for 1 hour (could be 1-3 hours).
5. Take out the cold sample and immediately centrifuge the sample for 5 minutes at 4,000×g. Take out 3 mL of the upper solvent layer and use a rotary evaporator to dry the sample in a 60-70° C. water bath under reduced pressure. Alternatively, the sample can be dried by blowing nitrogen gas in a 60-70° C. water bath. The dried sample can be stored at 20° C. or in a freezer for 1-2 days.
6. Add 1 mL of n-hexane to dissolve the sample and then add 0.8 mL of the solution [1×PBS: acetonitrile, 4:1, V/V], vortex for 1 minute.
7. Leave the tube open and heat the sample at 85° C. for 3 minutes
8. Centrifuge the sample for 10 minutes at 4,000×g.
9. Discard the upper organic layer. Use 50 μL of the lower aqueous layer for the assay.

### REFERENCES

The following references are incorporated herein by reference.
Abad, A., Moreno, M. J., and Montoya, A. 1999. Development of monoclonal antibody-based immunoassays to the N-methylcarbamate pesticide carbofuran. J. Agric. Food. Chem. 47:2475-2485.
Alderman, D. J. 1985. Malachite green: a review. J. Fish Dis. 8: 289-298.
Alderman, D. J., and Clifton-Hadley, R. S. 1993. Malachite green: a pharmacokinetic study in rainbow trout. J. Fish Dis. 16: 296-311.
Allen, J. L. 1990. Residues of malachite green in muscle, eggs, and fry from treated Atlantic salmon and chinook salmon. U.S. Fish Wildl. Serv. Invest. Fish Control, 101: 1-4.
Allen, J. L., and Hunn, J. B. 1986. Fate and distribution studies of some drugs used in aquaculture. Vet. Hum. Toxicol. 28(Suppl. 1):21-24.
Allen, J. L., Gofus, J. E., and Meinertz, J. R. 1994. Determination of malachite green residues in the eggs, fry, and adult muscle tissue of rainbow trout (Oncorhynchus mykiss). J. AOAC Int. 77:553-557.
Bauer, K., Dangschat, H., Knoppler, H.-O., and Neudergger, J. 1988. Uptake and excretion of malachite green by rainbow trout. Arch. Lebensmittelhyg. 39: 97-102.
Brandon, D. L., Holland, K. P., Dreas, J. S. 1998. Rapid screening for benzimidazole residues in bovine liver J. Agri. Food. Chem. 46:3653-3656.
Clegg, B. S., Stephenson, G. R., and Hall, J. C. 1999. Development of an enzyme-linked immunosorbent assay for the detection of glyphosate. J. Agric. Food. Chem. 47:5031-5037.
Clifton-Hadley, R. S., and Alderman, D. J. 1987. The effects of malachite green upon proliferative kidney disease. J. Fish Dis. 10: 101-107.
Culp, S. J., and Beland, F. A. 1996. Malachite green: a toxicological review. J. Am. Coll. Toxicol. 1996, 15: 219-238.
Culp, S. J., Beland, F. A., Heflich, R. H., Bensson, R. W., Blankenship, L. R., Webb, .P J., Mellick, P. W., Trotter, R. W., Sholton, S. D., Greenlees, K. J., and Manjanatha, M. G. 2002. Mutagenicity and carcinogenicity in relation to DNA adduct formation in rats fed leucomalachite green. Mutat. Res. 55- 506-507.
Fernandes, C., Lalitha, V. S., and Rao, K. V. K. 1991. Enhancing effect of malachite green on the development of hepatic preneoplastic lesions induced by N-nitrosodiethylamine in rats. Carcinogenesis, 12: 839-845.
Fessard, V., Godard, T., Huert, S., Mourot, A., and Poul, J. M. 1999. Mutagenicity of malachite and leucomalachite green in vitro tests. J. Appl. Toxicol. 19: 421-430.
Kasuga, Y., Hishida, M., Tanahashi, N., and Arai, M. 1992. Studies on disappearance of malachite green in cultured rainbow trout. J. Food Hyg. Soc. Jpn. 33: 539-542.
Kietzmann, M., Hapke, H.-J., Beeke, M., and Stehle, S. 1990. Problems in fish treatment with malachite green. Dtsch. Tieraerztl. Wochenschr. 97: 290-293.
Manz, D., Togel, J., Nilz, J., and Lutz, F. 1991. Studies of the use of therapeutic products in fish farming. Tieraerztl. Umsch. 46: 543-549.
Meyer, F. P., and Jorgenson, T. A. 1983. Teratological and other effects of malachite green on development of rainbow trout and rabbits. Trans. Am. Fish. Soc. 112: 818-824.
Nakagawa, M., Murata, K., Shimokawa, T., Honda, T., Kojima, S., and Uchiyama, M. 1984. Determination of residual methylene blue and malachite green in muscle and liver of rainbow trout and eel. Eisei Kagaku, 30: 301-308.
Plakas, S. M., El Said, K. R., Stehly, G. R., and Roybal, J. E. 1995. Optimization of a liquid chromatographic method for the determination of malachite green and its metabolites in fish tissues. J. AOAC Int. 78: 1388-1394.
Poe, W. E., and Wilson, R. P. 1983. Absorption of malachite green by channel catfish. Prog. Fish-Cult. 45:228-229.
Roybal, J. E., Pfenning, A. P., Munns, R. K., Holland, D. C., Hurlbut, J. A., and Long, A. R. 1995. Determination of malachite green and its metabolite, leucomalachite green, in catfish (Ictalurus punctatus) tissue by liquid chromatography with visible detection. J. AOAC Int. 78: 453-457.
Rushing, L. G., and Bowman, M. C. 1980. Determination of crystal violet in animal feed, human urine, and wastewater by high pressure liquid chromatography.J. Chromatogr. Sci. 18:224-232.
Rushing, L. G., and Hansen, E. B. 1997.Confirmation of malachite green, crystal violet and their leuco analogs in catfish and trout tissue by high-performance liquid chromatography utilizing electrochemistry with ultraviolet-visible diode array detection and fluorescence detection. J. Chromatogr. B. 700:223-231.
Rushing, L. G., Webb, S. F., and Thompson, H. C.1995. Determination of leucogentian violet and gentian violet in catfish tissue by high-performance liquid chromatography with visible detection. J. Chromatogr. B. 674:125-131.
Sagar, K., Smyth, M. R., Wilson, J. G., and McLaughlin, K. 1994. High-performance liquid chromatographic determination of the triphenylmethane dye, malachite green, using amperometric detection at a carbon fibre microelectrode. J. Chromatogr. A, 659: 329-336.
Sanborn, J. R., Gee, S. J., Gilman, S. D., Sugawara, Y., Jones, A. D., Rogers, J., Szurdoki, F., Stanker, L. H., Stoutamire, D. W., and Hammock, B. D. 1998. Hapten synthesis and antibody development for polychlorinated dibenzo-p-dioxin immunoassays. J. Agric. Food Chem. 46:2407-2416.
Wang, S., Allan, R. D., Skerritt, J. H., and Kennedy, I. R. 1999. Development of a compound-specific ELISA for flufenoxuron and an improved class-specific assay for benzoylphenylurea insect growth regulators. J. Agric. Food. Chem. 47:3416-3424.
Werth, G. 1958. Disturbances of the hereditary pattern and production of tumors by experimental tissue anoxia. Arzneim.-Forsch. 8: 735-744.
Werth, G., and Boiteux, A. 1968a. Toxicity of triphenylmethane dyes. Malachite green as an uncoupling agent of oxidative phosphorylation in vivo and in vitro. Arch. Toxicol. 23: 82-103.
Werth, G., and Boiteux, A. 1968b. The biologic activity of malachite green. 6. The detoxication of malachite green in the organism by the formation of leucomalachite green. Arzneim.-Forsch. 18: 39-42.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An antigen of Malachite Green Chromatic conjugating with antigenic poly(amino acid) via a linker R4 or any of R2, R3, R5, R6, wherein each R2, R3, R4, R5, and R6 are independently a linking group of 0 to 8 carbon atoms, 0-16 hydrogen atoms, 0 to 3 nitrogen atom bonded to carbon, 0-4 oxygen atom bonded to carbon and 0-1 sulfur bonded to carbon, and wherein the conjugating number of Malachite Green derivatives per poly (amino acid) is at least 1 but no greater than the molecular weight of the poly (amino acid) divided by 500.

2. Antigens according to claim 1, wherein the linker group R4 is —N═C═S, which is conjugated with antigenic poly(amino acid), and wherein the conjugating number of Malachite Green derivatives per poly (amino acid) is at least 1 but no greater than the molecular weight of the poly (amino acid) divided by 1500.

3. Antibodies produced by method of claim 1 that can detect and quantify Malachite Green Chromatic, Leucomalachite Green and Carbinol.

4. An antibody-based assay to detect Malachite Green Chromatic, Leucomalachite Green and Carbinol in a sample derived from food, feed, animals, animal excreta, human tissue, human excreta, plants, soil, air or water.

5. A method according to claim 4 for detecting Malachite Green Chromatic, Leucomalachite Green and Carbinol in a said sample by subjecting the said sample to an immunosorbent assay comprising: incubating said sample with the antibodies and detecting said bound Malachite Green Chromatic.

6. Methods of detecting and quantifying total Malachite Green Chromatic in a said sample using the procedure of claim 4 by converting Leucomalachite Green and Carbinol to Malachite Green Chromatic.

7. A kit for detecting or quantifying Malachite Green Chromatic, Leucomalachite Green and Carbinol in a sample derived from food, feed, animals, animal excreta, human tissue, human excreta, plants, soil, air or water.

8. The kit of claim 7 comprising antibodies reactive to Malachite Green Chromatic.

9. The kit of claim 7 further comprising a standard.

10. The kit of claim 7 further comprising a wash solution.

11. The kit of claim 7 further comprising reagents to detect Malachite Green Chromatic antibodies.

12. The kit of claim 7 further comprising a microtiter plate coated with Malachite Green Chromatic, Leucomalachite Green and Carbinol.

13. The kit according to claim 7 further comprising a buffer comprising of Sodium Acetate.

14. The kit according to claim 7 further comprising a buffer with a pH that is between 5 to 8.

15. Method for extracting Malachite Green Chromatic, Leucomalachite Green and Carbinol from the said sample in which during the said extraction procedure, the sample is incubated at a temperature that allows for the differential freezing of the aqueous layer from the organic layer.

16. The method according to claim 15 in which a solid aqueous layer forms while a liquid organic layer is maintained and is done by incubating the said sample at or near −20° C.
